(19) United States
(12) Patent Application Publication
(10) Pub. No.: US 2025/0125032 A1
(43) Pub. Date: Apr. 17, 2025

(54) DIGITALLY GUIDED PREHAB EXPERIENCES

(71) Applicant: DEEPWELL DTX, Las Vegas, NV (US)

(72) Inventors: Ryan J. Douglas, Seattle, WA (US); Samuel Robert Browd, Mercer Island, WA (US)

(73) Assignee: DEEPWELL DTX, Las Vegas, NV (US)

(21) Appl. No.: 19/000,211

(22) Filed: Dec. 23, 2024

Related U.S. Application Data
(63) Continuation-in-part of application No. 18/842,938, filed on Aug. 30, 2024.
(63) Continuation-in-part of application No. PCT/US23/69442, filed on Jun. 29, 2023.
(60) Provisional application No. 63/614,361, filed on Dec. 22, 2023.

(51) Int. Cl. G16H 20/30

(52) U.S. Cl. CPC G16H 20/30

(57) ABSTRACT

Apparatus and associated methods relate to automatically generate an immersive digital prehabilitation package (IDPP) to a user device based on a dynamically determined fitness metric associated with the user device. In an illustrative example, an adaptive personalized prehabilitation system (APPS) may, in response to an activation signal, retrieve a fitness metric and a prehabilitation experience associated with a user associated with the user device. The APPS, for example, may determine a mental fitness and a physical fitness of the user based on the fitness metric. For example, the APPS may adaptively generate a target state based on the fitness of the user and the prehabilitation experience, and the current state of the user. The APPS, for example, may generate the IDPP configured to induce the user to progress from a current state to the adaptive target state. Various embodiments may advantageously increase prehabilitation efficiency and/or reduce post-experience interventions.

### CROSS REFERENCE TO RELATED APPLICATIONS

This application is a continuation in part of and claims the benefit of U.S. application Ser. No. 18/842,938, titled “TREATMENT CONTENT DELIVERY AND PROGRESS TRACKING SYSTEM,” filed by Ryan J. Douglas, on Aug. 30, 2024, which is a national stage application of International Application Serial No. PCT/US2023/063720, titled “TREATMENT CONTENT DELIVERY AND PROGRESS TRACKING SYSTEM,” filed by Ryan J. Douglas, on Mar. 3, 2023, which claims the benefit of U.S. Provisional Application Ser. No. 63/268,905, titled “FDA-Compliant Therapeutic Game Selection and Delivery Platform,” filed by Ryan J. Douglas, on Mar. 4, 2022, U.S. Provisional Application Ser. No. 63/362,497, titled “Digital Platform for Delivery And Tracking of DTX Video Games,” filed by Ryan J. Douglas, on Apr. 5, 2022, U.S. Provisional Application Ser. No. 63/363,639, titled “Media Delivery Tool for Self-Assessment of Physical and Mental State,” filed by Ryan J. Douglas, on Apr. 26, 2022, and U.S. Provisional Application Ser. No. 63/366,521, titled “Therapeutic Game Selection and Delivery Engine,” filed by Ryan J. Douglas, on Jun. 16, 2022.

This application is also a continuation in part of and claims the benefit of International Application Serial No. PCT/US2023/069442, titled “DYNAMICALLY NEURO-HARMONIZED AUDIBLE SIGNAL FEEDBACK GENERATION,” filed by Ryan J. Douglas, et al., on Jun. 9, 2023, which claims the benefit of U.S. Provisional Application Ser. No. 63/367,235, titled “DEEPWELL MUSIC EXPERIENCE,” filed by Michael S. Wilson, et al., on Jun. 29, 2022, and U.S. Provisional Application Ser. No. 63/485,626, titled “Computer-Implemented Engagement and Therapeutic Mechanisms,” filed by Michael S. Wilson, et al., on Feb. 17, 2023.

This application also claims the benefit of U.S. Provisional Application Ser. No. 63/614,361, titled “DIGITALLY GUIDED PREHABILITATION EXPERIENCES,” filed by Ryan J. Douglas, on Dec. 22, 2024.

This application incorporates the entire contents of the foregoing application(s) herein by reference.

### TECHNICAL FIELD

Various embodiments relate generally to prehabilitation systems including dynamically generated immersive applications based on personalized metrics.

### BACKGROUND

Various digital technologies have been developed to facilitate communication between patients and healthcare providers, allowing for continuous monitoring, information exchange, and treatment management. Platforms such as mobile applications, telehealth systems, and wearable devices enable patients to report symptoms, track recovery progress, and receive real-time feedback from medical professionals. These communication tools have proven effective in improving engagement and allowing healthcare providers to make timely adjustments to treatment plans, ensuring better outcomes for patients undergoing medical interventions.

Additionally, digitally delivered content has been used to keep patients informed and educated throughout their treatment journeys. Mobile applications, websites, and connected devices deliver personalized content such as instructional videos, interactive guides, and educational articles tailored to specific conditions or procedures. This content assists patients in understanding the expected outcomes, step-by-step procedural information, and recovery milestones. By providing timely and targeted information, digital tools can help patients navigate their treatment processes with increased clarity and confidence.

Methods aimed at reducing emotional stress before and after surgery increasingly rely on articles, guided videos, and immersive content delivery. Preoperative digital resources, such as relaxation-focused videos, guided meditations, and educational materials, help prepare patients mentally by alleviating anxiety associated with surgical procedures. Similarly, postoperative content tailored to recovery phases can offer support through stress reduction techniques, mindfulness exercises, and coping strategies. By incorporating visual and auditory tools, these methods aim to foster emotional resilience and improve patient well-being during the preoperative and recovery periods.

### SUMMARY

Apparatus and associated methods relate to automatically generate an immersive digital prehabilitation package (IDPP) to a user device based on a dynamically determined fitness metric associated with the user device. In an illustrative example, an adaptive personalized prehabilitation system (APPS) may, in response to an activation signal, retrieve a fitness metric and a prehabilitation experience associated with a user associated with the user device. The APPS, for example, may determine a mental fitness and a physical fitness of the user based on the fitness metric. For example, the APPS may adaptively generate a target state based on the fitness of the user and the prehabilitation experience, and the current state of the user. The APPS, for example, may generate the IDPP configured to induce the user to progress from a current state to the adaptive target state. Various embodiments may advantageously increase prehabilitation efficiency and/or reduce post-experience interventions.

Various embodiments may achieve one or more advantages. For example, some embodiments may advantageously reduce medical inventions (e.g., surgical intervention, pharmaceutical intervention, psychological therapy) required for the user. Some embodiments may, for example, advantageously provide live therapies for prehabilitation. For example, some embodiments may advantageously prepare a user to be better able to sustain and/or quicker to recover from a surgical situation. Some embodiments may, for example, advantageously handle the side effects of a pharmaceutical on a greater level. For example, some embodiments may advantageously reduce side effects of a therapy. Some embodiments may, for example, advantageously reduce a need for postoperative pain control. For example, some embodiments may advantageously improve endurance of consequence of the experience. Some embodiments may, for example, advantageously reduce intervention in a recovery stage after, for example, a surgery. For example, some embodiments may advantageously ensure an adaptive prehabilitation program the patient's recovery progress and readiness for subsequent interventions. Some embodiments may, for example, advantageously enable users to mentally rehearse activities, triggering cortical reorganization and measurable improvements in readiness. For example, some embodiments may advantageously follow and track the user through a live experience(s). Some embodiments may, for example, advantageously reduce an effort including time to induce the user to progress from the current state to the adaptive target state. For example, some embodiments may advantageously select adaptively a course of target states aligning with the patient's capabilities and overall recovery goals.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary Adaptive Personalized Prehabilitation System (APPS) employed in an illustrative use-case scenario.

FIG. 2 is a block diagram depicting an exemplary APPS.

FIG. 3 is a block diagram depicting an exemplary data input output diagram of an exemplary APPS.

FIG. 4 is a block diagram depicting an exemplary prehabilitation modalities selection model.

FIG. 5 is a flowchart illustrating an exemplary APPS initialization method.

FIG. 6 is a flowchart illustrating an exemplary APPS runtime method.

Like reference symbols in the various drawings indicate like elements.

### DETAILED DESCRIPTION OF ILLUSTRATIVE EMBODIMENTS

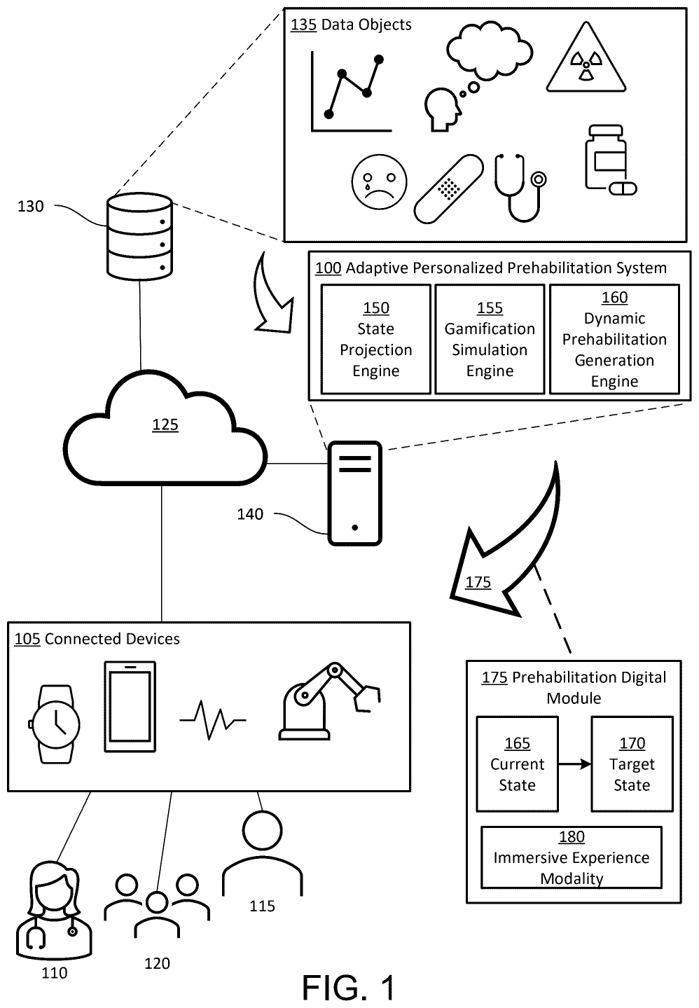

FIG. 1 depicts an exemplary Adaptive Personalized Prehabilitation System (APPS 100) employed in an illustrative use-case scenario. In the depicted example, device(s) 105 are communicably coupled to a network 125. The device(s) 105 may, for example, include personal devices. For example, the personal devices may include computing devices (computer, smartphone). The personal devices may, for example, include wearables (e.g., smartwatch, fitness tracker). The device(s) 105 may, for example, include medical devices. For example, the medical devices may include monitors (e.g., external monitors, embedded monitors) and/or actuators (e.g., imaging devices, therapy delivery devices, notification devices). The device(s) 105 may, for example, include systems (e.g., social media networks, content delivery platforms, research network systems, weather prediction networks, news/events feeds). For example, the systems may include servers and/or other connected devices (e.g., internet of things (IOT) devices). The device(s) 105 may be operated by and/or operably coupled to one or more users. For example, the users may include healthcare providers 110. The users may, for example, include patients 115 and/or related persons (e.g., caretakers, family, friends). The users may, for example, include other persons 120 (e.g., groups of persons).

The network 125 is communicably coupled to a computing device 140. For example, the computing device 140 may include a computer(s). In some embodiments, the computer(s) may, for example, be implemented as a server(s). For example, the computing device 140 may be implemented in a distributed computing network. The APPS 100 may, for example, be implemented on the computing device 140. For example, the APPS 100 may be operating on one or more processor(s) of the computing device 140.

The APPS 100 is communicably coupled to one or more data stores 130. For example, the one or more data stores 130 may include internal data stores. The one or more data stores 130 may, for example, include external data stores (e.g., as depicted). The one or more data stores 130 may contain one or more data objects 135. For example, the one or more data objects 135 may include structured data. The one or more data objects 135 may, for example, include unstructured data. In some implementations, by way of example and not limitation, the one or more data objects 135 may include patient reports (e.g., structured, unstructured such as in natural language). The one or more data objects 135 may include healthcare provider observations. The one or more data objects 135 may include data retrieved from electronic health record (EHR) system(s). The one or more data objects 135 may include data objects (e.g., weather reports, event data) retrieved from external providers. The one or more data objects 135 may, for example, include data from hardware (e.g., medical devices) such as, for example, one or more of the device(s) 105. For example, the data from hardware may include data from hospital devices (e.g., imaging, therapeutic delivery). The data from hardware may include fitness data, for example, such as from personal devices, such as wearables and/or smartphones. The data from hardware may, for example, include data from personal medical devices (e.g., implanted medical devices, external medical devices). The medical devices may, by way of example and not limitation, include monitoring and/or therapeutic delivery devices. For example, the data may include any data received from the device(s) 105.

In this example, the APPS 100 may generate and deliver prehabilitation modalities to users (e.g., the healthcare providers 110, the other persons 120, the patients 115) based on the one or more data objects 135. In some implementations, the APPS 100 may generate, within an application of the device(s) 105, digital modalities to prepare the patient for a therapeutic experience. For example, the therapeutic experience may include a surgical experience. For example, the therapeutic experience may include a pharmaceutical experience. For example, the therapeutic experience may include a health conditioning experience (e.g., a mental conditioning experience, a physical conditioning experience). For example, the physical conditioning experience may include weight loss programs. For example, the mental conditioning experience may include mental health programs.

In this example, the APPS 100 includes a state projection engine 150, a gamification simulation engine 155, and a dynamic prehabilitation generation engine 160. As an illustrative example, the APPS 100 may be preparing a user for a surgical procedure. For example, the dynamic prehabilitation generation engine 160 may be configured to induce the user from a current state 165 identified by the state projection engine 150 to a target state 170 by generating a prehabilitation digital module (PDM 175). For example, at the target state 170, the user may be expected to respond in a desired manner (e.g., medically and/or physically better prepared to endure the surgical procedure). In some examples, the target state 170 may include biometrics of a user indicating a higher likelihood of recovering faster from the surgical procedure. For example, the target state may include a physiologically shift. Various embodiments may advantageously reduce medical inventions (e.g., surgical intervention, pharmaceutical intervention, psychological therapy) required for the user. The PDM 175 may include an immersive experience modality 180 generated by the gamification simulation engine 155. For example, the immersive experience modality 180 may include immersive experiences in gaming and/or simulation.

In some implementations, the immersive experience modality 180 may include live experiences of using a medical device and/or experiencing a therapeutic experience. For example, some embodiments may advantageously provide live therapies for prehabilitation. Prehabilitation may, for example, precondition a user (e.g., patient) for a therapeutic and/or medical intervention (e.g., a surgery). For example, the prehabilitation may prepare a user to be more robust and/or more capable to manage that therapy.

For example, prehabilitation may advantageously prepare a user to be better able to sustain and/or quicker to recover from a surgical situation. Prehabilitation may, for example, prepare a user to more advantageously handle the side effects of a pharmaceutical on a greater level. Prehabilitation may prepare a user to precondition their body (e.g., mentally, physically) in a way that a lower dose may be therapeutic. For example, prehabilitation may make a user's body more bioavailable to accept the therapeutic. For example, prehabilitation may advantageously reduce side effects of a therapy.

In a conventional medical environment, a prehabilitation system may involve a patient to visit a doctor periodically (e.g., weekly, monthly) to apply medical interventions (e.g., dietary control, in-person fitness training, psychological consultation). In some examples, the frequency of the visit, especially in connection to the costs for each visit, may be limited.

Using the APPS 100, for example the prehabilitation may, for example, be performed through video games and/or other media (e.g., books, audio video) generated by the dynamic prehabilitation generation engine 160. In some implementations, for example, live media (e.g., a virtual event) may advantageously be used to provide prehabilitation. For example, some embodiments may deliver prehabilitation in hospital environments. Some embodiments may deliver prehabilitation in museum environments. Some embodiments may deliver prehabilitation in installation environments including places where additional benefits to the user using the PDM 175 may be delivered (e.g., to create immersive digital and physical experiences).

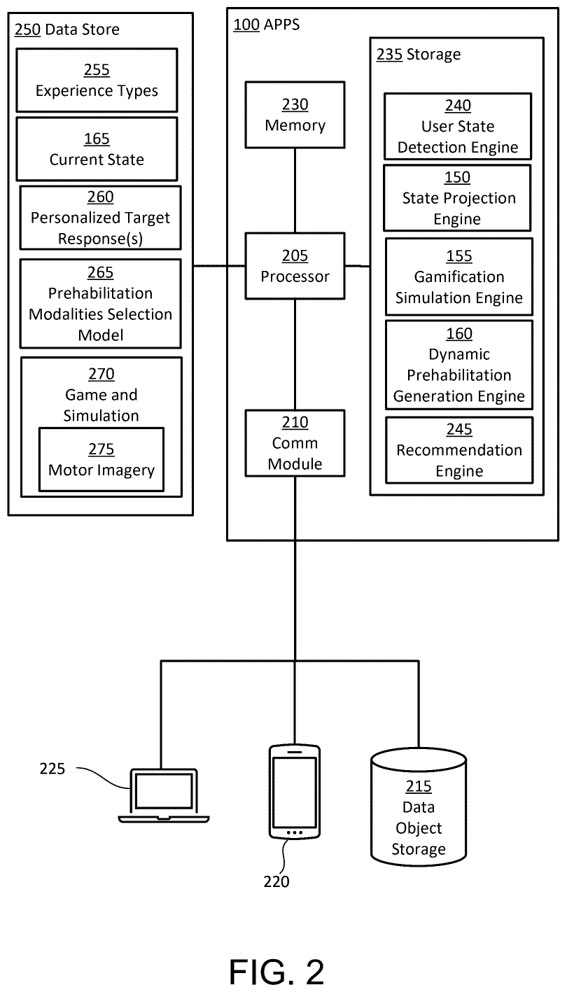

FIG. 2 is a block diagram depicting an exemplary APPS. The APPS 100 includes a processor 205. The processor 205 may, for example, include one or more processing units. The processor 205 is operably coupled to a communication module 210. The communication module 210 may, for example, include wired communication. The communication module 210 may, for example, include wireless communication. In the depicted example, the communication module 210 is operably coupled to a data object storage 215, a mobile device 220, and a computer device 225. For example, the data object storage 215 may store the one or more data objects 135. For example, the mobile device 220 and/or the computer device 225 may be used by a user of the APPS 100. For example, a client program of the APPS 100 may be installed in the mobile device 220 and/or the computer device 225. For example, the client program may be interconnected to one or more applications in the mobile device 220 and/or computer device 225 to receive user data (e.g., based on user behavior in other applications).

The processor 205 is operably coupled to a memory module 230. The memory module 230 may, for example, include one or more memory modules (e.g., random-access memory (RAM)). The processor 205 includes a storage module 235. The storage module 235 may, for example, include one or more storage modules (e.g., non-volatile memory). In the depicted example, the storage module 235 includes a user state identification engine 240, the state projection engine 150, the gamification simulation engine 155, the dynamic prehabilitation generation engine 160, and a recommendation engine 245. For example, the user state identification engine 240 may be configured to detect, based on the one or more data stores 130 from the data object storage 215 and a therapeutic experience, a current user state. For example, the gamification simulation engine 155 and/or the dynamic prehabilitation generation engine 160 may generate the immersive experience modality 180 and the PDM 175, respectively to the mobile device 220 and/or the data object storage 215.

For example, the recommendation engine 245 may generate a recommendation (e.g., for health planning, for intervention planning) to a user based on the current state 165 of the user. As an illustrative example, the recommendation engine 245 may recommend the user (e.g., and/or a medical personnel associated with the user) to schedule a surgery procedure when the current state 165 indicates that the user is ready.

The processor 205 is further operably coupled to a data store 250. The data store 250 includes experience types 255, the current state 165, a personalized target response(s) (PTR 260), a prehabilitation modalities selection model (PMSM 265), and a Game and Simulation 270. For example, the experience types 255 may include various experiences applicable in the APPS 100. For example, the experience types 255 may include various surgeries to be performed to a patient. For example, the experience types 255 may include various pharmaceutical programs applicable to the patients. For example, the experience types 255 may include mental and/or physical programs to be participated in by the patient.

The current state 165, for example, may be generated based on biometrics and other patient information by the user state identification engine 240. In some implementations, the dynamic prehabilitation generation engine 160 may generate the PTR 260 as a function of an experience associated with a user and the current state 165 of the user. For example, the PTR 260 may include physical thresholds and/or psychological thresholds of the user to be considered prepared for the experience. For example, the PTR 260 may include a baseline for pain threshold response. For example, based on the baseline, the gamification simulation engine 155 may generate the immersive experience modality 180 having destressing modalities configured to alter pain control (e.g., by introducing and teaching pain control mechanisms). For example, the immersive experience modality 180 may advantageously reduce a need for postoperative pain control.

In some implementations, the immersive experience modality 180 may include the target state 170. For example, the PMSM 265 may include training sessions. For example, the training session may include an immersive experience to walk through a target experience. For example, the Game and Simulation 270 may induce a heightened pain threshold.

As shown, the Game and Simulation 270 includes a motor imagery 275. For example, the motor imagery 275 may induce the user to imagine one or more physical activities to be performed. For example, through the induced imagination, a mental fitness and a physical fitness of the user may be triggered to progress towards the PTR 260. As an illustrative example without limitation, the motor imagery 275 may include information configured to enhance understanding of a surgery when the surgery is to be performed to the user. For example, the information may include a collective information of other users having similar experiences (e.g., going through the same surgery) to deliver shared experiences with a similar state of the user (e.g., based on the current state 165). In some implementations, the motor imagery 275 may represent new experience-associated stories generated by an artificial intelligence model based on the shared experience. For example, the new experience-associated story may advantageously reduce an ambiguity of the user to an experience to advantageously improve endurance of consequence of the experience. In some examples, the new experience-associated story may advantageously reduce intervention in a recovery stage after, for example, a surgery.

For example, the gamification simulation engine 155 may apply the PMSM 265 to select one or more games and/or simulations to be delivered to the user. For example, the PMSM 265 may be trained (e.g., configured) to select the motor imagery 275 with a highest likelihood of recovery during a post-operation stage.

In some implementations, the dynamic prehabilitation generation engine 160 may analyze a progression trend of a user. For example, the dynamic prehabilitation generation engine 160 may generate the progression trend as a function of a progression of the current state 165 over time. Based on the progression trend, the recommendation engine 245 may select to entrain a user to transition into a new experience. For example, the recommendation engine 245 may, based on a user's good response (e.g., based on a gait measurement) in a walking exercise, generate a recommendation to the user and/or a medical personnel to adjust a prescription for the user. In some implementations, the recommendation engine 245 may signal a user to transition from the prehabilitation to the procedure when the current state of the user reaches a predetermined threshold associated with the experience.

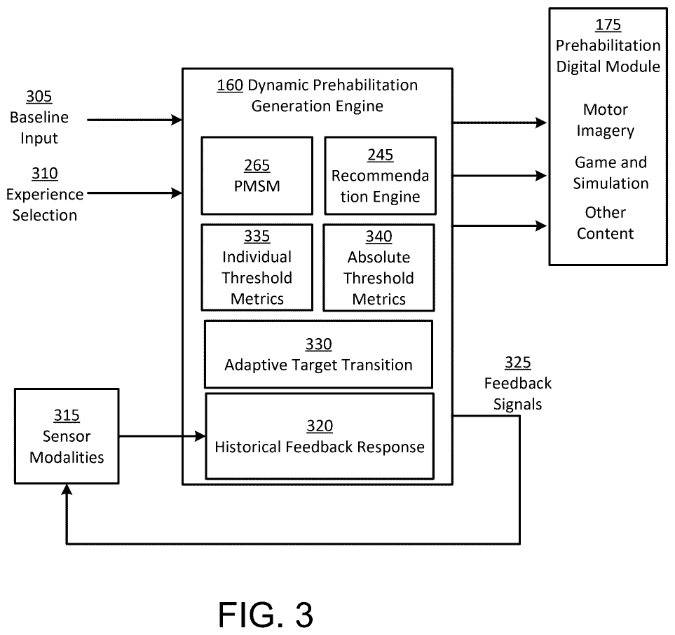

FIG. 3 is a block diagram depicting an exemplary data input output diagram of an exemplary dynamic prehabilitation generation engine. In this example, the dynamic prehabilitation generation engine 160 receives a baseline input 305 and an experience selection 310.

For example, the baseline input 305 includes any information related to the user. For example, the baseline input 305 may include a social history of the user. For example, the baseline input 305 may include a family history of the user. For example, the baseline input 305 may include a mental fitness metric. For example, the baseline input 305 may include a physical fitness metric. For example, the baseline input 305 may include a financial fitness metric.

For example, the experience selection 310 may include one of the experience types 255. In some implementations, the baseline input 305 may be generated from the data object storage 215. For example, the baseline input 305 may include other metrics related to a likelihood of success of the experience selection 310. For example, the baseline input 305 may include a historic metric of psychosocial emotional history of the user.

In this example, the dynamic prehabilitation generation engine 160 receives input from sensor modalities 315. For example, the sensor modalities 315 may include sensors of a mobile device (e.g., a smart phone). The sensor modalities 315 may, for example, include sensors of external wearables. In various embodiments, the sensor modalities 315 may receive personal data including location data, foot data, palms data, medication data, pumps, medication data, medication response data. In this example, the dynamic prehabilitation generation engine 160 includes a historical sensor response 320 to store data received from the sensor modalities 315. For example, the dynamic prehabilitation generation engine 160 may determine a change during a medical intervention (e.g., a pharmaceutical experience) process as a function of historical sensor response 320.

In this example, the sensor modalities 315 may also be receiving a feedback signal 325 of a user based on the PDM 175 generated for a user. For example, the recommendation engine 245 may, based on the historical sensor response 320 generate a signal to recommend a lower amount of drug dose for pain management prehabilitation to determine a result of the PDM 175.

The dynamic prehabilitation generation engine 160 may further integrate data from neural prosthetics and implanted sensors, for example, to enhance preparedness analysis and therapeutic planning. For example, data from sensors implanted during previous surgical interventions may be used to monitor recovery and assess readiness for future procedures. In this example, the dynamic prehabilitation generation engine 160 includes an adaptive target transition engine (ATTE 330). For example, the ATTE 330 may be configured to analyze a trend in the historical sensor response 320 with respect to the experience selection 310. By analyzing trends in sensor data over time using the historical sensor response 320, the ATTE 330 may, based on a patient's physiological response to various therapy and/or adapt prehabilitation programs, generate adaptive new targets for the patient based on the experience selection 310. This continuous monitoring and feedback enable personalized trajectory adjustments may advantageously ensure an adaptive prehabilitation program the patient's recovery progress and readiness for subsequent interventions. In cases involving complex or iterative procedures (e.g., orthopedic surgeries, skin grafting for burn recovery) the ATTE 330 and the recommendation engine 245 may assist healthcare providers to determine intervention timings and/provide automatic evaluation of a patient's capacity to tolerate additional procedures.

For example, the immersive experience modality 180 may induce the user to be involved in a level of visuospatial interference. For example, in one target state, the immersive experience modality 180 may be targeted at reducing anxiety and/or stress of a user. After the target state is reached, for example, the ATTE 330 may generate a new target. Based on the new target, for example, the gamification simulation engine 155 may generate the immersive experience modality 180 to induce the user to be involved with a narrative. For example, the narrative may include a story configured to visualize a reason for undergoing the therapeutic experience.

For example, based on the feedback signal 325, the dynamic prehabilitation generation engine 160 may analyze the user's response to prior experiences and adjust the immersive experience modality 180 to align with the user's evolving readiness and capabilities. The feedback signal 325 may include physiological data, behavioral data, and/or mental state metrics gathered from the user during prehabilitation. Using this data, the system may refine the immersive experiences to ensure the user progresses toward the desired therapeutic readiness.

In a next level, the immersive experience modality 180 may introduce a challenging condition. This condition could be specifically designed to include multiple levels of accomplishment, encouraging the user to build confidence and perceive themselves as capable. These accomplishments may serve as thresholds that indicate higher probabilities of success or improved outcomes for specific therapeutics. At this stage, the simulation may include in-app activities and/or immersive personal experiences, for example, involving external users and/or environment to complete individual tasks. Some experiences, for example, may influence the user's autonomic functioning (e.g., stress response, anxiety reduction, motivation enhancement).

For example, the immersive experience modality 180 may integrate cognitive-behavioral therapy, physical motion tracking, and guided motor imagery. Motor imagery, based on a first-person perspective, may advantageously enable users to mentally rehearse activities, triggering cortical reorganization and measurable improvements in readiness. While not identical to physical activity, for example, some mental imagery may provide proportional benefits. The APPS 100 may leverage this technique in gaming and/or in guided immersive experiences to create repetitive and reproducible routines for prehabilitation to promote user readiness.

After the PDM 175 is delivered, for example, the state projection engine 150 may (e.g., periodically, continuously) measure a physiological and/or mental change of the user to determine whether prehabilitation has been sufficiently achieved. In some implementations, the APPS 100 may include an interconnectivity with one or more external applications to monitor, for example, how a user plays games, and/or performs other actions. For example, the state projection engine 150 may, based on the one or more data stores 130 received from the device(s) 105, measure a change (e.g., measured) of thought patterns, activity patterns, and/or socialization patterns in preparation for an immersive experience. The immersive experience may, for example, be configured as a further preparation for the therapeutic itself. For example, the app may guide users through pre-experiences (e.g., as part of ‘pre’ prehabilitation) before the user deploy (e.g., or be delivered) the immersive experience modality 180. In some implementations, the APPS 100 may advantageously follow and track the user through a live experience(s). The app may, for example, measure how the user has changed to that point, and then monitor the user after they leave that experience. For example, the app may determine and/or notify (e.g., the user) when the user is ready for the next level of prehabilitation experience and/or for a therapeutic level, and/or should do more of the same work again.

The APPS 100 may also track (e.g., using the historical sensor response 320) users during live experiences, measuring their progress and determining readiness for the next stage of prehabilitation or the therapeutic experience. For example, the recommendation engine 245 may notify users when they are prepared to advance to the next level, or it may recommend repeating earlier steps for further reinforcement.

The dynamic prehabilitation generation engine 160 includes an individual threshold metrics 335 and an absolute threshold metrics 340. The dynamic prehabilitation generation engine 160 may assess a patient's readiness before surgery by comparing the feedback signal 325 to the individual threshold metrics 335 and/or the absolute threshold metrics 340. For example, the individual threshold metrics 335 may be dynamically generated based on the feedback signal 325 and the baseline input 305. For example, the individual threshold metrics 335 may allow the dynamic prehabilitation generation engine 160 to adaptively generate a personalized benchmark for readiness. As an illustrative example, the dynamic prehabilitation generation engine 160 may generate an individual psychological threshold for surgery based on a progression of walking steps by inferring a higher confidence in the patient.

For example, the absolute threshold metrics 340 may include an aggregated data from a group of outcomes to define minimum readiness criteria for a selected therapeutic intervention.

In some embodiments, the recommendation engine 245 may combine the individual threshold metrics 335 and the absolute threshold metrics 340 to determine whether a patient has achieved cognitive, mental, or physical levels necessary to proceed with the selected therapeutic experience. For example, a patient may need to meet a certain cardiovascular endurance level (e.g., the absolute threshold metrics 340) and/or demonstrate reduced stress (e.g., the individual threshold metrics 335) to qualify for surgery.

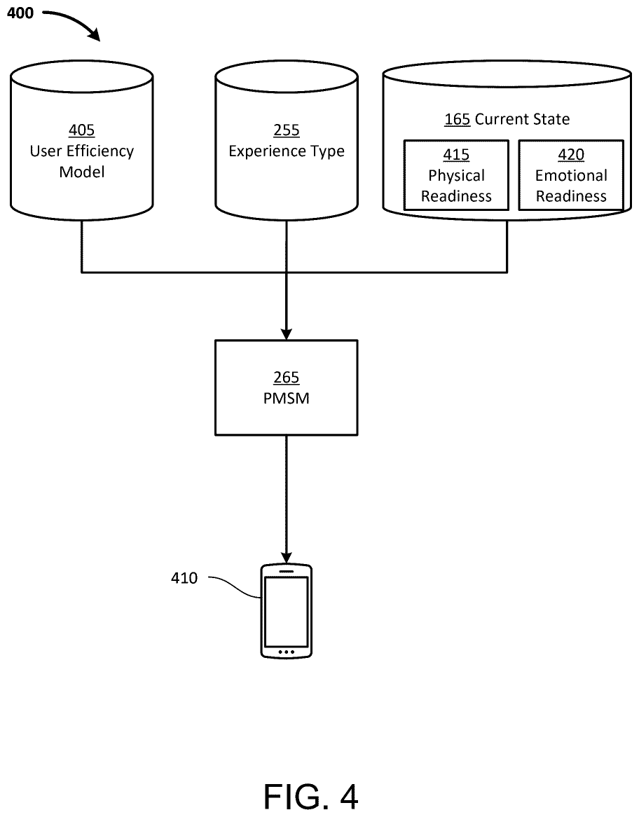

FIG. 4 is a block diagram depicting an exemplary prehabilitation modalities selection model. In this example, a PDM selection system 400 includes the PMSM 265. For example, the PDM selection system 400 may be used by the dynamic prehabilitation generation engine 160 to select the PDM 175 to be delivered to a user. As shown, the dynamic prehabilitation generation engine 160 may apply data from a user efficiency model 405, the experience types 255, and the current state 165 to the PMSM 265. For example, the PDM 175 may be generated to be delivered to a personal device 410 of the user.

For example, the user efficiency model 405 may dynamically assess and adapt to individual user preferences to enhance effectiveness or increase probability of success. For example, the user efficiency model 405 may include individualized stimuli and modalities identified to receive better responses from the user. For example, some users may find music more effective than video experiences. For example, some users may respond better to visual elements like flowers or animals. In some implementations, the user efficiency model 405 may capture and evaluate these unique preferences. For example, the PMSM 265 may, based on the user preference to select (e.g., in a bespoke manner) to generate the PDM 175 (e.g., the simulation 270).

Over time, in some implementations, the user efficiency model 405 may include a comprehensive understanding of the user through repeated interactions and collected data. For example, the APPS 100 may iteratively learn and improve the user efficiency model 405 to identify key triggers indicating when a user is ready to advance to the therapeutic or rehabilitation stage. For example, the ATTE 330 may apply the current state 165 to the user efficiency model 405 to determine when the user has achieved an acceptable level (e.g., based on a predetermined and/or an adaptively generated threshold) of readiness for a therapeutic intervention. Various embodiments may advantageously reduce an effort including time to induce the user to progress from the current state to the adaptive target state.

In this example, the current state 165 includes a physical readiness 415 and an emotional readiness 420. For example, the PMSM 265 may be configured to select the PDM 175 based on the physical readiness 415 and the emotional readiness 420 with respect to a selected experience. For example, the physical readiness 415 may include metrics including cardiovascular endurance, gait assessment, pain threshold levels, and other physical biometrics. In some examples, the emotional readiness 420 may assess factors including anxiety reduction, stress levels, and/or motivation.

For example, if a patient demonstrates high pain threshold levels and positive outcomes during prehabilitation, the PDM selection system 400 may determine that a more aggressive course of treatment could lead to faster recovery. By analyzing the physical readiness 415 and the emotional readiness 420, the PMSM 265 may adaptively generate the PDM 175 with respect to the preparatory phase, a planned therapeutic, and/or a planned rehabilitation trajectories (e.g., generated by the state projection engine 150). For example, the PDM selection system 400 may advantageously select adaptively a course of target states aligning with the patient's capabilities and overall recovery goals. Accordingly, for example, the PDM selection system 400 may allow for a personalized and intermediate outcome driven digital therapeutic delivery.

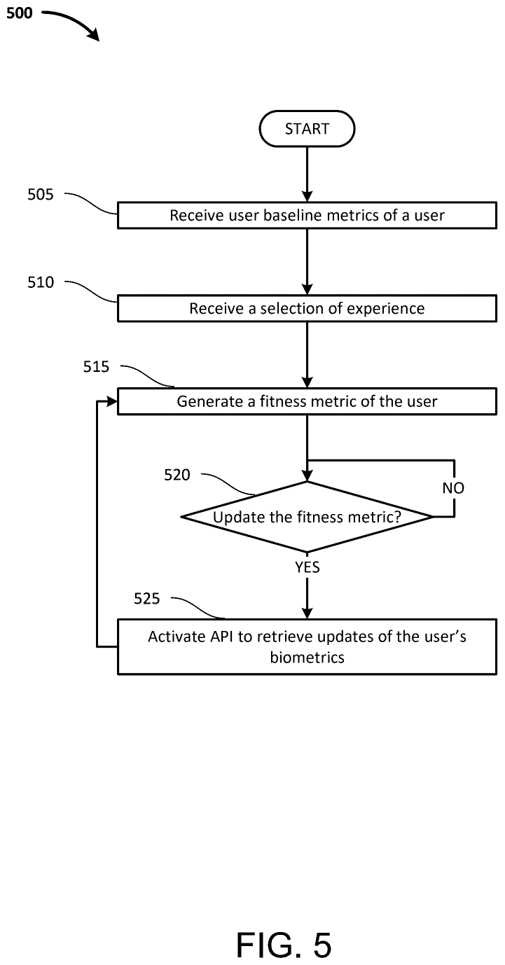

FIG. 5 is a flowchart illustrating an exemplary APPS initialization method 500. For example, the method 500 may be performed by the dynamic prehabilitation generation engine 160 to update or initialize a user within the APPS 100. In this example, the method 500 begins in step 505 when baseline metrics of a user are received. For example, the user state identification engine 240 may receive metrics such as cardiovascular endurance, gait assessment, and mental fitness from the device(s) 105, including wearable devices and personal medical devices.

In step 510, a selection of experience is received. For example, the PMSM 265 may receive an input corresponding to a specific therapeutic experience, such as a surgical preparation program, from a user interface of the personal device 220.

In step 515, a fitness metric of the user is generated. For example, the state projection engine 150 may process the baseline metrics and the selected experience to determine the fitness metric (e.g., associated with the current state 165, the emotional readiness 420, the 425//), which includes physical readiness 415 and emotional readiness 420. The state projection engine 150 may access data from the one or more data stores 130 to generate these metrics.

At a decision point 520, it is determined whether the fitness metric of the user needs to be updated. For example, the recommendation engine 245 may evaluate the feedback signal 325 to determine if changes in the user's biometrics or readiness require a recalibration of the fitness metric. If no update is required, the step 520 is repeated.

If an update is required, in step 525, an API (application programming interface) is activated to retrieve updates to the user's biometrics, and the step 515 is repeated. For example, the communication module 210 may activate the API to collect real-time biometric data from the sensor modalities 315, such as heart rate, step count, or stress levels, to refine the fitness metric.

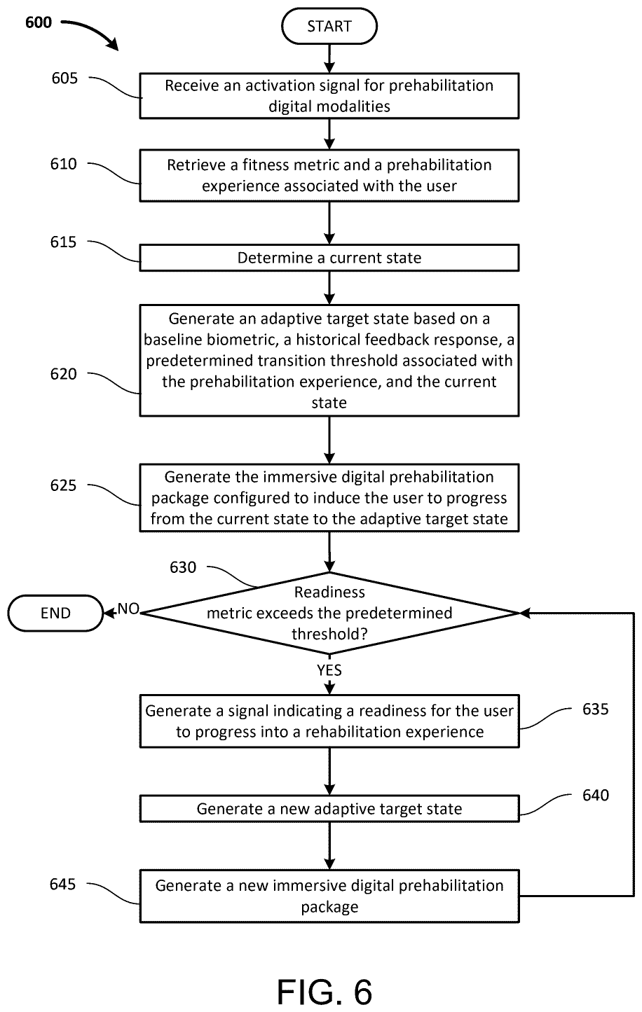

FIG. 6 is a flowchart illustrating an exemplary APPS runtime method 600. For example, the method 600 may be performed by the dynamic prehabilitation generation engine 160 within the APPS 100. In this example, the method 600 begins in step 605 when an activation signal for prehabilitation digital modalities is received. For example, the communication module 210 may receive the activation signal based on user input or a predefined schedule.

In step 610, a fitness metric and a prehabilitation experience associated with the user are retrieved. For example, the state projection engine 150 may access the fitness metrics (e.g., cardiovascular endurance, gait assessment) and the experience selection 310 from the data object storage 215.

In step 615, a current state of the user is determined. For example, the user state identification engine 240 may analyze the retrieved fitness metrics and associated experience data to generate the current state 165, including physical readiness 415 and emotional readiness 420.

In step 620, an adaptive target state is generated based on a baseline biometric, a historical feedback response, a predetermined transition threshold associated with the prehabilitation experience, and the current state. For example, the ATTE 330 may apply the historical sensor response 320 and feedback signal 325 to determine an adaptive target state for the user's therapeutic goals.

In step 625, an immersive digital prehabilitation package is generated, configured to induce the user to progress from the current state to the adaptive target state. For example, the gamification simulation engine 155 may generate the immersive experience modality 180, including tailored activities and visualizations, based on the target state parameters.

At a decision point 630, it is determined whether the readiness metric exceeds the predetermined threshold. For example, the recommendation engine 245 may compare the readiness metrics of the user, such as physiological and psychological thresholds, to the predefined thresholds associated with the prehabilitation experience. If the readiness metric does not exceed the threshold, the method 600 ends.

If the readiness metric exceeds the threshold, in step 635, a signal is generated indicating readiness for the user to progress into a rehabilitation experience. For example, the recommendation engine 245 may notify the user or healthcare provider through the personal device 220.

In step 640, a new adaptive target state is generated. For example, the ATTE 330 may determine the next target state based on the user's progression and updated readiness metrics. In step 645, a new immersive digital prehabilitation package is generated, and the step 630 is repeated. For example, the gamification simulation engine 155 may design a new package tailored to the user's next adaptive target state, ensuring continued progress toward therapeutic goals.

Although various embodiments have been described with reference to the figures, other embodiments are possible.

Although an exemplary system has been described with reference to the figures, other implementations may be deployed in other industrial, scientific, medical, commercial, and/or residential applications.

In various embodiments, some bypass circuits implementations may be controlled in response to signals from analog or digital components, which may be discrete, integrated, or a combination of each. Some embodiments may include programmed, programmable devices, or some combination thereof (e.g., PLAs, PLDs, ASICs, microcontroller, microprocessor), and may include one or more data stores (e.g., cell, register, block, page) that provide single or multi-level digital data storage capability, and which may be volatile, non-volatile, or some combination thereof. Some control functions may be implemented in hardware, software, firmware, or a combination of any of them.

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Although an example of a system, which may be portable, has been described with reference to the above figures, other implementations may be deployed in other processing applications, such as desktop and networked environments.

Temporary auxiliary energy inputs may be received, for example, from chargeable or single use batteries, which may enable use in portable or remote applications. Some embodiments may operate with other DC voltage sources, such as (nominal) batteries, for example. Alternating current (AC) inputs, which may be provided, for example from a 50/60 Hz power port, or from a portable electric generator, may be received via a rectifier and appropriate scaling. Provision for AC (e.g., sine wave, square wave, triangular wave) inputs may include a line frequency transformer to provide voltage step-up, voltage step-down, and/or isolation.

Although particular features of an architecture have been described, other features may be incorporated to improve performance. For example, caching (e.g., L1, L2, . . . ) techniques may be used. Random access memory may be included, for example, to provide scratch pad memory and or to load executable code or parameter information stored for use during runtime operations. Other hardware and software may be provided to perform operations, such as network or other communications using one or more protocols, wireless (e.g., infrared) communications, stored operational energy and power supplies (e.g., batteries), switching and/or linear power supply circuits, software maintenance (e.g., self-test, upgrades), and the like. One or more communication interfaces may be provided in support of data storage and related operations.

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and CD-ROM and DVD-ROM disks. The processor and the memory can be supplemented by, or incorporated in, ASICs (application-specific integrated circuits).

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device. The display device may, for example, include an LED (light-emitting diode) display. In some implementations, a display device may, for example, include a CRT (cathode ray tube). In some implementations, a display device may include, for example, an LCD (liquid crystal display). A display device (e.g., monitor) may, for example, be used for displaying information to the user. Some implementations may, for example, include a keyboard and/or pointing device (e.g., mouse, trackpad, trackball, joystick), such as by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces and/or memory circuits fabricated on a silicon substrate including various integrated circuits (e.g., FPGAs, ASICs), or some combination thereof. In some embodiments, the module(s) may involve execution of preprogrammed instructions, software executed by a processor, or some combination thereof. For example, various modules may involve both hardware and software.

In some aspects, the techniques described herein relate to a system including: a data store including a program of instructions; and, a processor operably coupled to the data store such that, when the processor executes the program of instructions, the processor causes operations to be performed to automatically generate an immersive digital prehabilitation package to a user device based on a dynamically determined fitness metric associated with the user device, the operations including: in response to an activation signal, retrieve a fitness metric and a prehabilitation experience associated with a user associated with the user device, wherein the prehabilitation experience includes an intervention associated with the user; determine a current state including a mental fitness and a physical fitness of the user based on the fitness metric; generate an adaptive target state based on a baseline biometric, a historical feedback response, a predetermined transition threshold associated with the prehabilitation experience, and the current state of the user; and, generate the immersive digital prehabilitation package configured to induce the user to progress from the current state to the adaptive target state, wherein the immersive digital prehabilitation package includes: a readiness metric of the user to perform the intervention; and, a motor imagery configured to induce the user to imagine one or more physical activities to be performed, such that the mental fitness and the physical fitness are triggered to progress towards a predetermined threshold required to perform the intervention.

In some aspects, the techniques described herein relate to a system, further includes a rehabilitation experience, wherein the operations further include: determine whether the readiness metric exceeds the predetermined transition threshold; generate a signal indicating a readiness for the user to progress into the rehabilitation experience associated with the prehabilitation experience; upon receiving a confirmation signal, generate a new adaptive target state based on the baseline biometric, the historical feedback response, the rehabilitation experience, and the current state of the user; and, generate a new immersive digital prehabilitation package configured to induce the user to progress from the current state to the new adaptive target state.

In some aspects, the techniques described herein relate to a system, wherein the fitness metric includes a surgical experience, a pharmaceutical experience, a mental experience, and a physical experience.

In some aspects, the techniques described herein relate to a system, further includes identify a user preference, wherein the immersive digital prehabilitation package is selected by applying the user preference to an immersive digital prehabilitation package selection model, wherein the immersive digital prehabilitation package selection model is configured to reduce an effort including time to induce the user to progress from the current state to the adaptive target state.

In some aspects, the techniques described herein relate to a system, wherein the mental fitness includes a pain threshold response.

In some aspects, the techniques described herein relate to a system, wherein the predetermined transition threshold is generated as a function of the baseline biometric, a minimum threshold associated with the prehabilitation experience, and an aggregated threshold identified based on historical outcomes of individuals undergoing a same prehabilitation experience.

In some aspects, the techniques described herein relate to a computer-implemented method performed by at least one processor to automatically generate an immersive digital prehabilitation package to a user device based on a dynamically determined fitness metric associated with the user device, the method including: in response to an activation signal, retrieve a fitness metric and a prehabilitation experience associated with a user associated with the user device, wherein the prehabilitation experience includes an intervention associated with the user; determine a current state including a mental fitness and a physical fitness of the user based on the fitness metric; generate an adaptive target state based on a baseline biometric, a historical feedback response, a predetermined transition threshold associated with the prehabilitation experience, and the current state of the user; and, generate the immersive digital prehabilitation package configured to induce the user to progress from the current state to the adaptive target state, wherein the immersive digital prehabilitation package includes a motor imagery configured to induce the user to imagine one or more physical activities to be performed, such that the mental fitness and the physical fitness are triggered to progress towards a predetermined threshold required to perform the intervention.

In some aspects, the techniques described herein relate to a computer-implemented method, wherein the immersive digital prehabilitation package further includes a readiness metric of the user to perform the intervention.

In some aspects, the techniques described herein relate to a computer-implemented method, further includes: determine whether the readiness metric exceeds the predetermined transition threshold; generate a signal indicating a readiness for the user to progress into a rehabilitation experience associated with the prehabilitation experience; upon receiving a confirmation signal, generate a new adaptive target state based on the baseline biometric, the historical feedback response, the rehabilitation experience, and the current state of the user; and, generate a new immersive digital prehabilitation package configured to induce the user to progress from the current state to the new adaptive target state.

In some aspects, the techniques described herein relate to a computer-implemented method, wherein the fitness metric includes a surgical experience, a pharmaceutical experience, a mental experience, and a physical experience.

In some aspects, the techniques described herein relate to a computer-implemented method, further includes identify a user preference, wherein the immersive digital prehabilitation package is selected by applying the user preference to an immersive digital prehabilitation package selection model, wherein the immersive digital prehabilitation package selection model is configured to reduce an effort including time to induce the user to progress from the current state to the adaptive target state.

In some aspects, the techniques described herein relate to a computer-implemented method, wherein the mental fitness includes a pain threshold response.

In some aspects, the techniques described herein relate to a computer-implemented method, wherein the predetermined transition threshold is generated as a function of the baseline biometric, a minimum threshold associated with the prehabilitation experience, and an aggregated threshold identified based on historical outcomes of individuals undergoing a same prehabilitation experience.

In some aspects, the techniques described herein relate to a computer program product including a program of instructions tangibly embodied on a non-transitory computer readable medium wherein, when the instructions are executed on a processor, the processor causes digital modality generation operations to be performed to automatically generate an immersive digital prehabilitation package to a user device based on a dynamically determined fitness metric associated with the user device, the operations including: in response to an activation signal, retrieve a fitness metric and a prehabilitation experience associated with a user associated with the user device, wherein the prehabilitation experience includes an intervention associated with the user; determine a current state including a mental fitness and a physical fitness of the user based on the fitness metric; generate an adaptive target state based on a baseline biometric, a historical feedback response, a predetermined transition threshold associated with the prehabilitation experience, and the current state of the user; and, generate the immersive digital prehabilitation package configured to induce the user to progress from the current state to the adaptive target state, wherein the immersive digital prehabilitation package includes a motor imagery configured to induce the user to imagine one or more physical activities to be performed, such that the mental fitness and the physical fitness are triggered to progress towards a predetermined threshold required to perform the intervention.

In some aspects, the techniques described herein relate to a computer program product, wherein the immersive digital prehabilitation package further includes a readiness metric of the user to perform the intervention.

In some aspects, the techniques described herein relate to a computer program product, wherein the operations further includes: determine whether the readiness metric exceeds the predetermined transition threshold; generate a signal indicating a readiness for the user to progress into a rehabilitation experience associated with the prehabilitation experience; upon receiving a confirmation signal, generate a new adaptive target state based on the baseline biometric, the historical feedback response, the rehabilitation experience, and the current state of the user; and, generate a new immersive digital prehabilitation package configured to induce the user to progress from the current state to the new adaptive target state.

In some aspects, the techniques described herein relate to a computer program product, wherein the fitness metric includes a surgical experience, a pharmaceutical experience, a mental experience, and a physical experience.

In some aspects, the techniques described herein relate to a computer program product, further includes identify a user preference, wherein the immersive digital prehabilitation package is selected by applying the user preference to an immersive digital prehabilitation package selection model, wherein the immersive digital prehabilitation package selection model is configured to reduce an effort including time to induce the user to progress from the current state to the adaptive target state.

In some aspects, the techniques described herein relate to a computer program product, wherein the mental fitness includes a pain threshold response.

In some aspects, the techniques described herein relate to a computer program product, wherein the predetermined transition threshold is generated as a function of the baseline biometric, a minimum threshold associated with the prehabilitation experience, and an aggregated threshold identified based on historical outcomes of individuals undergoing a same prehabilitation experience.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. A system comprising:
a data store comprising a program of instructions; and,
a processor operably coupled to the data store such that, when the processor executes the program of instructions, the processor causes operations to be performed to automatically generate an immersive digital prehabilitation package to a user device based on a dynamically determined fitness metric associated with the user device, the operations comprising:
in response to an activation signal, retrieve a fitness metric and a prehabilitation experience associated with a user associated with the user device, wherein the prehabilitation experience comprises an intervention associated with the user;
determine a current state comprising a mental fitness and a physical fitness of the user based on the fitness metric;
generate an adaptive target state based on a baseline biometric, a historical feedback response, a predetermined transition threshold associated with the prehabilitation experience, and the current state of the user; and,
generate the immersive digital prehabilitation package configured to induce the user to progress from the current state to the adaptive target state, wherein the immersive digital prehabilitation package comprises:
a readiness metric of the user to perform the intervention; and,
a motor imagery configured to induce the user to imagine one or more physical activities to be performed, such that the mental fitness and the physical fitness are triggered to progress towards a predetermined threshold required to perform the intervention.

2. The system of claim 1, further comprises a rehabilitation experience, wherein the operations further comprise:
determine whether the readiness metric exceeds the predetermined transition threshold;
generate a signal indicating a readiness for the user to progress into the rehabilitation experience associated with the prehabilitation experience;
upon receiving a confirmation signal, generate a new adaptive target state based on the baseline biometric, the historical feedback response, the rehabilitation experience, and the current state of the user; and,
generate a new immersive digital prehabilitation package configured to induce the user to progress from the current state to the new adaptive target state.

3. The system of claim 1, wherein the fitness metric comprises a surgical experience, a pharmaceutical experience, a mental experience, and a physical experience.

4. The system of claim 1, further comprises identify a user preference, wherein the immersive digital prehabilitation package is selected by applying the user preference to an immersive digital prehabilitation package selection model, wherein the immersive digital prehabilitation package selection model is configured to reduce an effort comprising time to induce the user to progress from the current state to the adaptive target state.

5. The system of claim 1, wherein the mental fitness comprises a pain threshold response.

6. The system of claim 1, wherein the predetermined transition threshold is generated as a function of the baseline biometric, a minimum threshold associated with the prehabilitation experience, and an aggregated threshold identified based on historical outcomes of individuals undergoing a same prehabilitation experience.

7. A computer-implemented method performed by at least one processor to automatically generate an immersive digital prehabilitation package to a user device based on a dynamically determined fitness metric associated with the user device, the method comprising:
in response to an activation signal, retrieve a fitness metric and a prehabilitation experience associated with a user associated with the user device, wherein the prehabilitation experience comprises an intervention associated with the user;
determine a current state comprising a mental fitness and a physical fitness of the user based on the fitness metric;
generate an adaptive target state based on a baseline biometric, a historical feedback response, a predetermined transition threshold associated with the prehabilitation experience, and the current state of the user; and,
generate the immersive digital prehabilitation package configured to induce the user to progress from the current state to the adaptive target state, wherein the immersive digital prehabilitation package comprises a motor imagery configured to induce the user to imagine one or more physical activities to be performed, such that the mental fitness and the physical fitness are triggered to progress towards a predetermined threshold required to perform the intervention.

8. The computer-implemented method of claim 7, wherein the immersive digital prehabilitation package further comprises a readiness metric of the user to perform the intervention.

9. The computer-implemented method of claim 8, further comprises:
determine whether the readiness metric exceeds the predetermined transition threshold;
generate a signal indicating a readiness for the user to progress into a rehabilitation experience associated with the prehabilitation experience;
upon receiving a confirmation signal, generate a new adaptive target state based on the baseline biometric, the historical feedback response, the rehabilitation experience, and the current state of the user; and,
generate a new immersive digital prehabilitation package configured to induce the user to progress from the current state to the new adaptive target state.

10. The computer-implemented method of claim 7, wherein the fitness metric comprises a surgical experience, a pharmaceutical experience, a mental experience, and a physical experience.

11. The computer-implemented method of claim 7, further comprises identify a user preference, wherein the immersive digital prehabilitation package is selected by applying the user preference to an immersive digital prehabilitation package selection model, wherein the immersive digital prehabilitation package selection model is configured to reduce an effort comprising time to induce the user to progress from the current state to the adaptive target state.

12. The computer-implemented method of claim 7, wherein the mental fitness comprises a pain threshold response.

13. The computer-implemented method of claim 7, wherein the predetermined transition threshold is generated as a function of the baseline biometric, a minimum threshold associated with the prehabilitation experience, and an aggregated threshold identified based on historical outcomes of individuals undergoing a same prehabilitation experience.

14. A computer program product comprising a program of instructions tangibly embodied on a non-transitory computer readable medium wherein, when the instructions are executed on a processor, the processor causes digital modality generation operations to be performed to automatically generate an immersive digital prehabilitation package to a user device based on a dynamically determined fitness metric associated with the user device, the operations comprising:
in response to an activation signal, retrieve a fitness metric and a prehabilitation experience associated with a user associated with the user device, wherein the prehabilitation experience comprises an intervention associated with the user;
determine a current state comprising a mental fitness and a physical fitness of the user based on the fitness metric;
generate an adaptive target state based on a baseline biometric, a historical feedback response, a predetermined transition threshold associated with the prehabilitation experience, and the current state of the user; and,
generate the immersive digital prehabilitation package configured to induce the user to progress from the current state to the adaptive target state, wherein the immersive digital prehabilitation package comprises a motor imagery configured to induce the user to imagine one or more physical activities to be performed, such that the mental fitness and the physical fitness are triggered to progress towards a predetermined threshold required to perform the intervention.

15. The computer program product of claim 14, wherein the immersive digital prehabilitation package further comprises a readiness metric of the user to perform the intervention.

16. The computer program product of claim 15, wherein the operations further comprises:
determine whether the readiness metric exceeds the predetermined transition threshold;
generate a signal indicating a readiness for the user to progress into a rehabilitation experience associated with the prehabilitation experience;
upon receiving a confirmation signal, generate a new adaptive target state based on the baseline biometric, the historical feedback response, the rehabilitation experience, and the current state of the user; and,
generate a new immersive digital prehabilitation package configured to induce the user to progress from the current state to the new adaptive target state.

17. The computer program product of claim 14, wherein the fitness metric comprises a surgical experience, a pharmaceutical experience, a mental experience, and a physical experience.

18. The computer program product of claim 14, further comprises identify a user preference, wherein the immersive digital prehabilitation package is selected by applying the user preference to an immersive digital prehabilitation package selection model, wherein the immersive digital prehabilitation package selection model is configured to reduce an effort comprising time to induce the user to progress from the current state to the adaptive target state.

19. The computer program product of claim 14, wherein the mental fitness comprises a pain threshold response.

20. The computer program product of claim 14, wherein the predetermined transition threshold is generated as a function of the baseline biometric, a minimum threshold associated with the prehabilitation experience, and an aggregated threshold identified based on historical outcomes of individuals undergoing a same prehabilitation experience.
